(19) United States
(12) Patent Application Publication
(10) Pub. No.: US 2008/0014288 A1
(43) Pub. Date: Jan. 17, 2008

(54) Zinc-containing treatments for hyperphosphatemia

(76) Inventors: Chad C. Huval, Somerville, MA (US); Stephen Randall Holmes-Farley, Arlington, MA (US); Pradeep K. Dhal, Westford, MA (US); Edmund J. Sybertz, Sudbury, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/820,297

(22) Filed: Jun. 19, 2007

Related U.S. Application Data
(63) Continuation of application No. PCT/US05/47582, filed on Dec. 29, 2005.
(60) Provisional application No. 60/640,643, filed on Dec. 30, 2004.

(51) Int. Cl. A61K 33/30; A61K 31/315; A61P 7/00

(52) U.S. Cl. 424/641; 514/494

(57) ABSTRACT

A method of treating hyperphosphatemia in a subject comprises the step of administering to the subject an effective amount of a pharmaceutically acceptable zinc salt. A pharmaceutical composition comprises a pharmaceutically acceptable carrier; a pharmaceutically acceptable zinc salt; and a phosphate sequestrant. In one embodiment, the phosphate sequestrant is selected from a pharmaceutically acceptable lanthanum, calcium, magnesium and iron salt. In another embodiment, the phosphate sequestrant is an amine polymer, wherein the molar ratio of a zinc ion of the zinc salt to amine nitrogen atoms in the amine polymer is 0.1-3.0. The invention also includes a pharmaceutical composition comprising a pharmaceutically acceptable carrier; a pharmaceutically acceptable zinc salt; and an agent selected from the group consisting of a phosphate transport inhibitor, an HMG-CoA reductase inhibitor and an alkaline phosphatase inhibitor.

### RELATED APPLICATION

This application is a continuation of International Application No. PCT/US2005/047582, which designated the United States and was filed on Dec. 29, 2005, published in English, which claims the benefit of U.S. Provisional Application No. 60/640,643 filed on Dec. 30, 2004. The entire teachings of these applications are incorporated herein by reference.

### BACKGROUND OF THE INVENTION

People with inadequate renal function, hypoparathyroidism, or certain other medical conditions often have hyperphosphatemia, or elevated serum phosphate levels. Hyperphosphatemia, especially if present over extended periods of time, leads to severe abnormalities in calcium and phosphorus metabolism, often manifested by hyperparathyroidism, bone disease and calcification in joints, lungs, eyes and vasculature. For patients who exhibit renal insufficiency, elevation of serum phosphorus within the normal range has been associated with progression of renal failure and increased risk of cardiovascular events.

Oral administration of certain phosphate binders to bind intestinal phosphate and prevent absorption has also been suggested. Typical phosphate binders include calcium, aluminum and magnesium salts. More recently, lanthanum and iron salts have been used as phosphate binders. The major problem with the calcium therapy is the hypercalcemia that often results from absorption of high amounts of ingested calcium, which can cause serious side effects such as cardiac arrhythmias, renal failure, and skin and visceral calcification. Thus, frequent monitoring of serum calcium levels is required during therapy with calcium-based phosphate binders. Aluminum-based phosphate binders, such as Amphojel® aluminum hydroxide gel, have also been used for treating hyperphosphatemia. However, prolonged use of aluminum gels leads to accumulations of aluminum, and often to aluminum toxicity, accompanied by such symptoms as encephalopathy, osteomalacia, and myopathy. Other calcium and aluminum-free phosphate binders have drawbacks including the amount and frequency of dosing required to be therapeutically active.

Consequently, there is a need for an alternative therapy for hyperphosphatemia. In particular, a therapy with reduced daily dose volume would be more desirable in order to improve patient compliance.

### SUMMARY OF THE INVENTION

It has now been found that pharmaceutically acceptable zinc salts are efficient agents for reducing urinary phosphate levels. As shown in Example 1, for example, zinc oxide, zinc chloride, zinc acetate dihydrate, zinc glycinate monohydrate and zinc citrate dihydrate substantially reduce urinary phosphate levels in vivo. For example, the amount of phosphate excreted in the urine of a house Sprague Dawley (SD) rat dosed with 0.5% by weight of zinc oxide in Purina 5002 was 18.4% of control rats that did not undergo the zinc oxide therapy. Based on this discovery, novel methods of treating a subject with hyperphosphatemia and novel pharmaceutical compositions are disclosed herein.

In one embodiment, the present invention is a method of treating hyperphosphatemia in a subject. The method comprises the step of administering to the subject an effective amount of a pharmaceutically acceptable zinc salt.

In another embodiment, the present invention is a pharmaceutical composition comprising a pharmaceutically acceptable carrier; a pharmaceutically acceptable zinc salt; and an amine polymer, wherein the molar ratio of the zinc ion of the zinc salt to amine nitrogen atoms in the amine polymer is 0.1-3.0.

In yet another embodiment, the present invention is a pharmaceutical composition comprising a pharmaceutically acceptable carrier; a pharmaceutically acceptable zinc salt; and an agent selected from the group consisting of a pharmaceutically acceptable lanthanum salt, a pharmaceutically acceptable calcium salt, a pharmaceutically acceptable magnesium salt, a pharmaceutically acceptable iron salt, a phosphate transport inhibitor, an HMG-CoA reductase inhibitor and an alkaline phosphatase inhibitor.

The pharmaceutical compositions and methods disclosed herein can reduce urinary phosphate levels, and thus can be used for treating hyperphosphatemia, chronic renal failure, hypoparathyroidism, or diseases associated with disorders of phosphate metabolism.

### DETAILED DESCRIPTION OF THE INVENTION

Pharmaceutically acceptable zinc salts are utilized in the present invention. As used herein, a “pharmaceutically acceptable zinc salt” means a zinc compound which does not cause unacceptable side effects at the dosages which are being administered. The pharmaceutically acceptable zinc salts can be water-soluble or water-insoluble.

It is to be understood that a “pharmaceutically acceptable zinc salt” may encompass different polymorphs of the pharmaceutically acceptable zinc salt. The term “polymorph” refers to solid crystalline forms of a compound, each of which may exhibit different physical, chemical or spectroscopic properties.

The “pharmaceutically acceptable zinc salt” may also include various solvates of the pharmaceutically acceptable zinc salt, which include a stoichiometric or non-stoichiometric amount of solvent, e.g., water or organic solvent, bound by non-covalent intermolecular forces.

Preferred pharmaceutically acceptable zinc salts have a high weight percentage of zinc, and/or have a high density. These zinc salts can minimize daily dose volume. Examples of pharmaceutically acceptable zinc salts suitable for the invention include zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate). Another example includes poly(zinc acrylate). Optionally, a mixture of pharmaceutically acceptable zinc salts can be used in the disclosed methods and pharmaceutical compositions.

In one embodiment, the term pharmaceutically acceptable zinc salt excludes zinc salts of fatty acids. A “fatty acid” as used herein means a saturated, unsaturated, branched or linear, aliphatic carboxylic acid having 15-50 carbon atoms (e.g., 15-25 carbon atoms or 17-20 carbon atoms), such as stearic acid, arachidonic acid or linoleic acid.

Examples of preferred pharmaceutically acceptable zinc salts in the present invention include zinc oxide, zinc acetate, zinc citrate, zinc carbonate and zinc tartrate, more preferably zinc oxide.

The present invention also includes a pharmaceutical composition comprising a pharmaceutically acceptable carrier; a pharmaceutically acceptable zinc salt; and a phosphate sequestrant. Optionally, the pharmaceutical composition of the invention comprises a mixture of pharmaceutically acceptable zinc salts. Suitable examples of the pharmaceutically acceptable zinc salts for the composition are as described above.

As used herein, the “phosphate sequesterant” means a pharmaceutically acceptable compound which binds phosphate. The phosphate sequestrant can be a calcium-, aluminum-, magnesium-, lanthanum- or iron-containing phosphate binder or a phosphate-binding amine polymer such as those disclosed in U.S. Pat. Nos. 5,496,545, 5,667,775 and 6,083,495 (the contents of which are incorporated herein by reference in their entirety). Preferably, the phosphate-binding polymer is an amine polymer, more preferably an aliphatic amine polymer.

Amine polymers are characterized by a repeat unit that includes at least one amine group. Amine groups can be part of the polymer backbone (e.g., a polyalkyleneimine such as polyethyleneimine), pendant from the polymer backbone (e.g., polyallylamine), or both types of amine groups can exist within the same repeat unit and/or polymer. Amine polymers include aliphatic amine polymers and aromatic amine polymers. The word “amine,” as used herein, includes primary, secondary and tertiary amines, as well as ammonium groups such as trialkylammonium.

Aromatic amine polymers comprise an aromatic amine moiety in one or more of the repeat units. An example of an aromatic amine polymer is poly(aminostyrene) (e.g., cholestyramine). 6

Aliphatic amine polymers are characterized by a repeat unit that includes at least one aliphatic amine group. An aliphatic amine polymer may be obtained by polymerizing an aliphatic amine monomer. An aliphatic amine is a saturated or unsaturated, straight-chained, branched or cyclic non-aromatic hydrocarbon having an amine substituent and optionally one or more additional substituents. An aliphatic amine monomer is an aliphatic amine comprising a polymerizable group such as an olefin. Suitable aliphatic amine polymers are described in U.S. Pat. Nos. 5,487,888, 5,496,545, 5,607,669, 5,618,530, 5,624,963, 5,667,775, 5,679,717, 5,703,188, 5,702,696, 5,693,675, 5,900,475, 5,925,379, 6,083,497, 6,177,478, 6,083,495, 6,203,785, 6,423,754, 6,509,013, 6,605,270, 6,726,905, 6,733,780 and 6,858,203 and U.S. Published Applications Nos. 2002/0159968 A1 and 2003/0086898 A1, the contents of which are incorporated herein by reference in their entireties.

An aliphatic amine polymer may be a homopolymer or a copolymer of one or more aliphatic amine monomers or a copolymer of one or more aliphatic amine monomers in combination with one or more monomers which do not comprise an amine are preferably inert and non-toxic. Examples of suitable monomers which do not comprise an amine include vinyl alcohol, acrylic acid, acrylamide, and vinylformamide. Alternatively, an aliphatic amine polymer can be a co-polymer of two or more different aliphatic amine monomers.

Examples of aliphatic amine polymers include polymers that have one or more repeat units selected from Formulas (1)-(6): 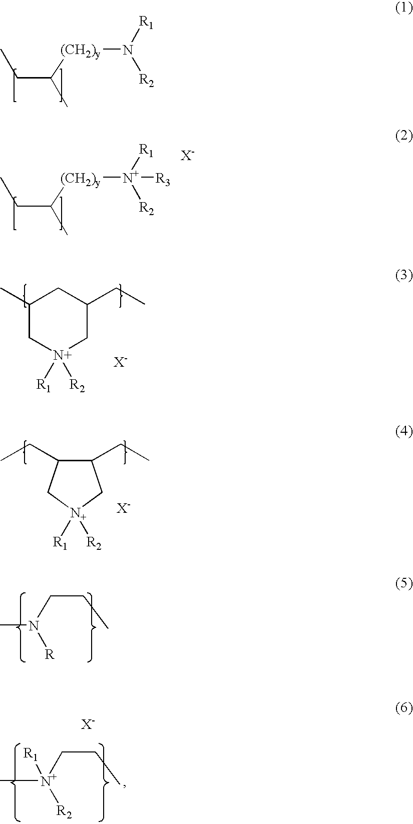
or a salt or copolymer thereof, where y is zero or an integer of one or more (e.g., between about one and about 10, preferably between one and four, more preferably one) and each R, R₁, R₂, and R₃, independently, is H, a substituted or unsubstituted alkyl group (e.g., having between 1 and 25 or between I and 5 carbon atoms, inclusive) or aryl (e.g., phenyl) group, and each X⁻ is an exchangeable negatively charged counterion.

Preferably, at least one of R, R₁, R₂, or R₃ is a hydrogen atom. More preferably, each of these groups is hydrogen.

The alkyl or aryl group, represented by R, R₁, R₂, and R₃, can carry one or more substituents. Suitable substituents include cationic groups, e.g., quaternary ammonium groups, or amine groups, e.g., primary, secondary or tertiary alkyl or aryl amines. Examples of other suitable substituents include hydroxy, alkoxy, carboxamide, sulfonamide, halogen, alkyl, aryl, hydrazine, guanidine, urea, poly(alkyleneimine) such as poly(ethylenimine), and carboxylic acid esters.

Preferably, an aliphatic amine polymer is a homopolymer, such as a homopolyallylamine polymer, a homopolyvinylamine polymer, a homopolydiallylamine polymer or a polyethyleneamine polymer, but can also be a co-polymer.

In one embodiment, the aliphatic amine polymer is a homopolymer or copolymer characterized by one or more repeat units of Structural Formula (7): 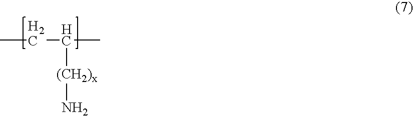
or a pharmaceutically acceptable salt thereof, where x is 0 or an integer between 1 and 4, preferably 1. The polymer represented by Structural Formula (7) is advantageously crosslinked by means of a crosslinking agent.

A preferred aliphatic amine polymer for use in the invention is a polyallylamine polymer, which is a polymer having repeat units from polymerized allylamine monomers. The amine group of an allylamine monomer can be unsubstituted or substituted with, for example, one or two C1-C10 straight chain or branched alkyl groups. These alkyl groups are optionally substituted with one or more hydroxyl, amine, halo, phenyl, amide or nitrile groups. Preferably, the aliphatic amine polymers that may be used in the present invention are polyallylamine polymers comprising repeat units represented by Structural Formula (8):

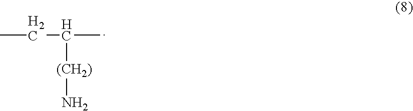

Polyallylamine polymers that may be used in the present invention may include copolymers comprising repeat units from two or more different polymerized allylamine monomers or with repeat units from one or more polymerized allylamine monomers and repeat units from one or more polymerized monomers which are not allylamines. Examples of suitable monomers which are not allylamines include acrylamide monomers, acrylate monomers, maleic acid, malimide monomers, vinyl acylate monomers and alkyl substituted olefines. Alternatively, other olefinic aliphatic amine monomers can be polymerized with an allylamine monomer. Preferably, however, the polyallylamine polymers used in the present invention comprise repeat units solely from polymerized allylamine monomers. More preferably, the polyallylamine polymers used in the present invention are homopolymers. Even more preferably, the polyallylamine polymers used in the present invention are homopolymers of repeat units represented by Structural Formula (8). Polyallylamine polymers used in the disclosed invention are preferably crosslinked polymers, more preferably crosslinked homopolymers.

Other examples of aliphatic amine polymers suitable for use in the invention are copolymers of diethylenetriamine, preferably crosslinked by means of a multifunctional crosslinking agent. Preferably, the aliphatic amine polymer is an epichlorohydrin-crosslinked copolymer of diethylenetriamine, such as colestipol.

In other embodiments, the amine polymers suitable for use in the invention can be a homopolymer or copolymer of polybutenylamine, polylysine, or polyarginine.

Amine polymers (aliphatic and aromatic amine polymers) are typically crosslinked with crosslinking agents. Preferably, the amine polymers are rendered water-insoluble by crosslinking such as with a crosslinking agent. Suitable crosslinking agents include those with functional groups which react with the amine group of the amine monomer. Alternatively, the crosslinking agent may contain two or more vinyl groups which undergo free radical polymerization with the amine monomer. In some cases the amine polymers are crosslinked after polymerization.

Examples of suitable crosslinking agents include diacrylates and dimethylacrylates (e.g., ethylene glycol diacrylate, propylene glycol diacrylate, butylene glycol diacrylate, ethylene glycol dimethacrylate, propylene glycol dimethacrylate, butylene glycol dimethacrylate, polyethyleneglycol dimethacrylate and polyethyleneglycol diacrylate), methylene bisacrylamide, methylene bismethacrylaamide, ethylene bisacrylamide, ethylene bismethacrylamide, ethylidene bisacrylamide, divinylbenzene, bisphenol A, the diglycidal ether of bisphenol A, pyromellitic dianhydride, toluene diisocyanate, ethylene diamine and dimethyl succinate, dimethacrylate, and bisphenol A diacrylate. Examples of preferred difunctional crosslinking agents include epichlorohydrin, 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, toluene diisocyanate, acryloyl chloride, and pyromellitic dianhydride. Epichlorohydrin is a most preferred crosslinking agent, because of its high availability and low cost. Epichlorohydrin is also advantageous because of its low molecular weight and hydrophilic nature, increasing the water-swellability and gel properties of the polyamine. Epichlorohydrin formns 2-hydroxypropyl crosslinking groups.

Other methods of inducing crosslinking on already polymerized materials include, but are not limited to, exposure to ionizing radiation, ultraviolet radiation, electron beams, radicals, and pyrolysis.

The level of crosslinking renders the crosslinked amine polymers insoluble and substantially resistant to absorption and degradation, thereby limiting the activity of the crosslinked amine polymers to the gastrointestinal tract, and reducing potential side-effects in the patient. Typically, the crosslinking agent is present in an amount 0.5-35% (such as 0.5-25%, 2.5-20% or 1-10%) by weight, based upon total weight of amine monomer plus crosslinking agent.

Typically, between about 3% and about 30% of the allylic nitrogen atoms are bonded to a crosslinking group, preferably between 6% and about 21%.

The amine polymers can also be further derivatized; examples include alkylated amine polymers, as described, for example, in U.S. Pat. Nos. 5,679,717, 5,607,669 and 5,618,530, the teachings of which are incorporated herein by reference in their entireties. Preferred alkylating agents include hydrophobic groups (such as aliphatic hydrophobic groups) and/or quaternary ammonium- or amine-substituted alkyl groups.

Non-crosslinked and crosslinked polyallylamine polymers and polyvinylamine polymers are generally known in the art and are commercially available. Methods for the manufacture of polyallylamine polymers and polyvinylamine polymers, and crosslinked derivatives thereof, are described in the above U.S. Patents. Patents by Harada et al., (U.S. Pat. Nos. 4,605,701 and 4,528,347), which are incorporated herein by reference in their entireties, also describe methods of manufacturing polyallylamine polymers and crosslinked polyallylamine polymers. A patent by Stutts et al., (U.S. Pat. No. 6,180,754) describes an additional method of manufacturing crosslinked polyallylamine polymers.

The molecular weight of amine polymers is not believed to be critical, provided that the molecular weight is large enough so that the amine polymers are substantially non-absorbed by the gastrointestinal tract. Typically, the molecular weight of amine polymers, preferably aliphatic amine polymers, is at least 1000. For example, the molecular weight can be from: about 1000 to about 5 million, about 1000 to about 3 million, about 1000 to about 2 million, or about 1000 to about 1 million.

The amine polymers used in the invention may be optionally protonated, and in one embodiment, include amine polymers in which less than 40%, for example, less than 30%, such as less than 20% or less than 10%, of the amine groups are protonated. In another embodiment, 35% to 45% of the amines are protonated (e.g., approximately 40%). An example of a suitably protonated amine polymer is sevelamer hydrochloride.

As described above, the amine polymer can be administered in the form of a pharmaceutically acceptable salt. The term “pharmaceutically acceptable salt” of an amine polymer refers to a salt of the amine polymer to be administered which is prepared from pharmaceutically acceptable non-toxic acids including inorganic acids, organic acids, solvates, hydrates, or clathrates thereof. Thus, the nitrogen group in the repeat unit of the amine polymer is protonated to create a positively charged nitrogen atom associated with a negatively charged counterion. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic(pamoic), methanesulfonic, ethanesulfonic, pantothenic, benzenesulfonic(besylate), stearic, sulfanilic, alginic, galacturonic, and the like.

Examples of suitable counterions include organic ions, inorganic ions, or a combination thereof. For instance, suitable counterions include halides (e.g., F⁻, Cl⁻, Br⁻ and I⁻), CH₃OSO₃⁻, HSO₄⁻, SO₄²⁻, HCO₃⁻, CO₃²⁻, acetate, lactate, succinate, propionate, oxalate, butyrate, ascorbate, citrate, dihydrogen citrate, tartrate, taurocholate, glycocholate, cholate, hydrogen citrate, maleate, benzoate, folate, an amino acid derivative, a nucleotide, a lipid, or a phospholipid. Preferred anions are Cl⁻, HCO₃⁻, CO₃²⁻, or a combination thereof (e.g., a mixed carbonate and bicarbonate salt, a mixed carbonate and chloride salt, or a mixed bicarbonate and chloride salt). The counterions can be the same as, or different from, each other. For example, the amine polymer can contain two or more different types of counterions.

In a preferred embodiment, the amine polymer used in the present invention is an epichlorohydrin-crosslinked polyallylamine polymer, such as sevelamer and colesevelam (see, for example, U.S. Pat. Nos. 6,423,754; 5,607,669; and 5,679,717, the contents of which are incorporated herein by reference). In a more preferred embodiment, the polyallylamine polymer is crosslinked with epichlorohydrin and between about 9% to about 30% (preferably about 15% to about 21%) of the allylic nitrogen atoms are bonded to a crosslinking group and the anion is chloride, carbonate or bicarbonate or a mixed salt thereof.

A particularly preferred amine polymer is polyallylamine hydrochloride crosslinked with about 9.0-9.8% w/w epichlorohydrin, preferably 9.3-9.5%, and is the active chemical component of the drug known as sevelamer HCl, sold under the tradename RENAGEL®. The structure is represented below: 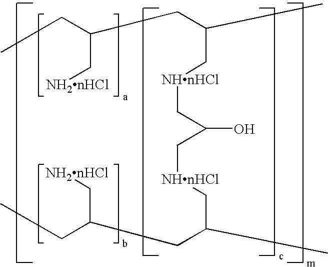
where:
the sum of a and b (the number of primary amine groups) is 9;
c (the number of crosslinking groups) is 1;
n (the fraction of protonated amines) is 0.4; and
m is a large number (to indicate extended polymer network).

Another particularly preferred amine polymer is a polyallylamine hydrochloride crosslinked with epichlorohydrin, and alkylated with 1-bromodecane and (6-bromohexyl)-trimethylammonium bromide, referred to as colesevelam HCl, and marketed in the United States as WELCHOL®.

In yet another particularly preferred embodiment, the amine polymer is a carbonate salt of sevelamer; a bicarbonate salt of sevelamer; a mixed carbonate and bicarbonate salt of sevelamer; or a mixed carbonate and chloride salt of sevelamer.

In other embodiments, a monovalent anionic source is mixed with a carbonate salt of an aliphatic amine polymer. Various examples of carbonate salts of the aliphatic amine polymer and monovalent anionic sources are disclosed in U.S. Provisional Application No. 60/624,001 “Aliphatic Amine Polymer Salts For Tableting” filed Nov. 1, 2004 and U.S. Provisional Application No. 60/628,752 “Aliphatic Amine Polymer Salts For Tableting” filed Nov. 17, 2004, the entire contents of which are incorporated herein by reference.

The monovalent anion comprises at least 0.01%, preferably 0.05%, more preferably a range of 0.01% to 2%, 0.05% to 1%, 0.08% to 0.5%, or 0.1% to 0.3% by weight of the combined weights of the carbonate salt of an aliphatic amine polymer and the monovalent anion source.

Examples of suitable monovalent anions include organic ions, inorganic ions, or a combination thereof, such as halides (Cl⁻, I⁻, F⁻ and Br⁻), CH₃OSO₃⁻, HSO₄⁻, acetate, lactate, butyrate, propionate, sulphate, citrate, tartrate, nitrate, sulfonate, oxalate, succinate or palmoate. Preferred monovalent anions are halides, most preferably chloride.

Also, the monovalent anion source can be a pharmaceutically acceptable acid, ammonium or metal salt of a monovalent anion. Preferably, the metal salt is not a zinc salt. Preferred examples of the monovalent anion source include sodium chloride and hydrochloric acid. In one preferred embodiment, the formulations of the invention comprise a carbonate salt of sevelamer and sodium chloride. In another preferred embodiment, the formulations of the invention comprise a carbonate salt of sevelamer and hydrochloric acid.

In yet another embodiment, when a carbonate salt of an aliphatic amine polymer is included in the invention, the monovalent anion source can be a monovalent anion salt of an aliphatic amine polymer comprising a repeat unit represented by Structural Formulas (1)-(8) above. In this embodiment, a monovalent anion salt of an aliphatic amine polymer and the carbonate salt of an aliphatic amine polymer can be physically mixed together. Alternatively, a single aliphatic amine polymer can comprise both carbonate and monovalent anions to form a mixed carbonate and monovalent anion salt of the single aliphatic amine polymer. When a monovalent anion salt of an aliphatic amine polymer and a carbonate salt of an aliphatic amine polymer are physically mixed together, the monovalent anion salt of an aliphatic amine polymer can be the same or a different aliphatic amine polymer as the aliphatic amine polymer carbonate salt.

When an amine polymer as described above is used in combination with a pharmaceutically acceptable zinc salt of the invention, the amine polymer and pharmaceutically acceptable zinc salt can be co-formulated in a single pharmaceutical composition, or alternatively co-administered in separate pharmaceutical compositions.

In some embodiments, the amine polymer, preferably an aliphatic amine polymer, and pharmaceutically acceptable zinc salt are co-formulated in a single pharmaceutical composition. The amine polymer and pharmaceutically acceptable zinc salt can be present in an admixture thereof Alternatively, the pharmaceutically acceptable zinc salt can be entrained within a crosslinked amine polymer, preferably a crosslinked aliphatic amine polymer, as described above. As used herein, the phrase “the pharmaceutically acceptable zinc salt entrained within a crosslinked amine polymer” means that the crosslinked amine polymer encaptures the pharmaceutically acceptable zinc salt, for example, within a pocket (or pockets) generated by crosslinking. A pharmaceutically acceptable zinc salt entrained with a crosslinked amine polymer, preferably a crosslinked aliphatic amine polymer, can be prepared by crosslinking an amine polymer as described above in the presence of a pharmaceutically acceptable zinc salt. For example, a polyallylamine polymer can be crosslinked by multifunctional crosslinking agent(s), such as epichlorohydrin, in the presence of zinc oxide to form a crosslinked polyallylamine polymer entraining zinc oxide. Various examples and preferred values for the amine polymers, crosslinking agents and pharmaceutically acceptable zinc salts are as described above. Typically, when a pharmaceutically acceptable zinc salt entrained with a crosslinked amine polymer, preferably a crosslinked aliphatic amine polymer, is employed, the crosslinking agent is present in an amount 0.5-35% (such as 0.5-30%, 2.5-30%, 5-25%, 5-20% or 5-15%) by weight, based upon total weight of amine monomer plus crosslinking agent.

When the amine polymer, preferably an aliphatic amine polymer, and a pharmaceutically acceptable zinc salt are formulated in a single pharmaceutical composition, typically, the zinc ion of the pharmaceutically acceptable zinc salt comprises 5-35%, such as 10-30%, 10-25%, 13-25%, 15-22% and 16-20%, by anhydrous weight of the pharmaceutical composition.

Alternatively, the zinc ion of the pharmaceutically acceptable zinc salt comprises 5-35%, such as 10-30%, 10-25%, 13-25%, 15-22% and 16-20%, by anhydrous weight of the combined weight of the zinc salt and the free base of the amine polymer. Herein, the term “the free base of the amine polymer” means the amine polymer not including any counter ion. When the quantity of zinc salt in the pharmaceutical composition is expressed in this fashion, it is to be understood that the amine polymer in the pharmaceutical composition can be unprotonated, partially protonated or completely protonated. However, the weight of the amine polymer is calculated assuming it is the corresponding free base amine polymer and that all of the nitrogen atoms in the amine polymer are free and not bound to any counter ions.

Alternatively, the pharmaceutically acceptable zinc salt is present in the pharmaceutical composition in an amount such that the molar ratio of the zinc ion of the pharmaceutically acceptable zinc salt to the total amine nitrogen atoms (protonated and unprotonated) of the polymer is 0.1-3.0, such as 0.4-3.0, 0.4-2.5, 0.8-2.0, 0.8-1.5 and 0.8-1.3. Preferably, the molar ratio is 1. This ratio is the quotient of moles of zinc ion of the pharmaceutically acceptable zinc salt to moles of nitrogen atom in the amine polymer. If present, nitrogen from a counter ion or cross-linker is included in the moles of the amine polymer.

Alternatively, the pharmaceutically acceptable zinc salt is present in the pharmaceutical composition in an amount such that the weight ratio of the zinc ion of the pharmaceutically acceptable zinc salt to the total nitrogen atoms of the amine polymer is 0.7-2.5, such as 0.7-2.0, 1.0-2.0 and 1.2-1.8. Preferably, the weight ratio is 1.57. This weight ratio is the quotient of grams of zinc ion to grams of nitrogen atoms in the amine polymer (but not the entire composition). Thus, nitrogen from a counter ion or cross-linker, if present, is included in the grams of the nitrogen atoms in the amine polymer.

Alternatively, the pharmaceutically acceptable zinc salt is present in the pharmaceutical composition in an amount such that the weight ratio of the zinc ion of the pharmaceutically acceptable zinc salt to the free base of the aliphatic amine polymer is 0.2-1.2, such as 0.2-1.0, 0.3-1.0, 0.3-0.8 and 0.3-0.5. Preferably, the weight ratio is 0.42. The term “the free base of the amine polymer” is as described above. Thus, this ratio is the quotient of grams of zinc ion to grams of amine polymer not including any weight from any counter ion in the amine polymer.

Other phosphate sequestrants suitable for use in the present invention include pharmaceutically acceptable lanthanum, calcium, aluminum, magnesium and iron salts, such as acetates, carbonates, oxides, hydroxides, citrates, alginates, and ketoacids. Calcium salts, including calcium carbonate, acetate (such as PhosLo® calcium acetate tablets), citrate, alginate, and ketoacids, have been utilized for phosphate binding. The ingested calcium combines with phosphate to form insoluble calcium phosphate salts such as Ca₃(PO₄)₂, CaHPO₄, or Ca(H₂PO₄)₂. Aluminium-based phosphate sequestrants, such as Amphojel® aluminium hydroxide gel, have also been used for treating hyperphosphatemia. These compounds complex with intestinal phosphate to form highly insoluble aluminium phosphate; the bound phosphate is unavailable for absorption by the patient. More recently iron and lanthanide salts have been used. The most commonly used lanthanide salt, lanthanum carbonate (Fosrenol®) behaves similarly to calcium carbonate. Other phosphate sequestrants suitable for use in the present invention include pharmaceutically acceptable magnesium salts. Various examples of pharmaceutically acceptable magnesium salts are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof. When referring to any of these phosphate sequestrants, it is to be understood that mixtures, polymorphs and solvates thereof are encompassed.

In some embodiments, a mixture of the phosphate sequestrants (e.g., a mixture of a pharmaceutically acceptable magnesium salt and an amine polymer, preferably an aliphatic amine polymer, or a mixture of a pharmaceutically acceptable iron salt and an amine polymer, preferably an aliphatic amine polymer) described above can be used in the invention in combination with the pharmaceutically acceptable zinc salts.

In another embodiment, the method and pharmaceutical composition of the invention are directed to a combination therapy of a pharmaceutically acceptable zinc salt in combination with a phosphate transport inhibitor, an HMG-CoA reductase inhibitor such as a statin, or an alkaline phosphatase inhibitor. Alternatively, a mixture of pharmaceutically acceptable zinc salts is employed together with a phosphate transport inhibitor, an HMG-CoA reductase inhibitor such as a statin, or an alkaline phosphatase inhibitor. Suitable pharmaceutically acceptable zinc salts for the therapy are as described above.

Suitable examples of phosphate transport inhibitors can be found in co-pending U.S. Application Publication Nos. 2004/0019113 and 2004/0019020 and WO 2004/085448, the entire teachings of each of which are incorporated herein by reference.

One example of a phosphate transport inhibitor which can be used in the present invention is a bis-aryl compound represented by Structural Formula (9) (described in U.S. Application Publication No. 2004/0019113):
Ar₁—W′—X′—Y′—Ar₂ (9),
or a pharmaceutically acceptable salt thereof. These phosphate transport inhibitors can be used in combination with a pharmaceutically acceptable zinc salt or a mixture of pharmaceutically acceptable zinc salts. Ar₁ and Ar₂ are independently substituted or unsubstituted aryl groups or an optionally substituted five membered or six membered non-aromatic heterocylic group fused to an optionally substituted monocylic aryl group (e.g., phenyl group). W′ and Y′ are independently a covalent bond or a C1-C3 substituted or unsubstituted alkylene group, (preferably a covalent bond or —CH₂—; more preferably a covalent bond). X′ is a heteroatom-containing functional group, an aromatic heterocyclic group, substituted aromatic heterocyclic group, non-aromatic heterocyclic group, substituted non-aromatic heterocyclic group, an olefin group or a substituted olefin group.

In another example, a phosphate transport inhibiting compound represented by Structural Formula (10) (described in U.S. Application Publication No. 2004/0019020): 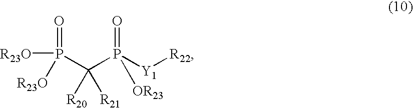
or a pharmaceutically acceptable salt or a prodrug thereof can be used in the invention in combination with a pharmaceutically acceptable zinc salt or a mixture of pharmaceutically acceptable zinc salts.

R₂₀ and R₂₁ are independently —H, an electron withdrawing group or a C₁-C₁₀ alkyl group. Preferably, R₂₀ and R₂₁ are independently —H or —F.

Y₁ is a covalent bond, a substituted methylene group, an unsubstituted methylene group, or —CR₂₀ R₂₁P(O)(OH)—. Preferably, Y₁ is a covalent bond or —CHQ-, wherein Q is —H, —F or a lower alkyl group. Y₁ can also be —CV₂—, where V is —H, —F or a lower alkyl group, preferably —F.

R₂₂ is a hydrocarbyl group optionally comprising one or more amine, ammonium, ether, thioether or phenylene linking groups, a substituted hydrocarbyl group optionally comprising one or more amine, ammonium, ether, thioether or phenylene linking groups, a heteroaryl group, a substituted heteroaryl group or a phenyl group substituted with one or more groups selected from —Cl, —Br, —F, —CN, —NO₂, —OR^{a}, —O(halogenated lower alkyl) such as —OCF₃, —N(R^{a})₂, —COOR^{a}, —CON(R^{a})₂, —COR^{a}, —S(O)R^{a}, —S(O)₂R^{a}, —S(O)₂N(R^{a})₂, —NR^{a}S(O)₂R^{a}, —NR^{a}COR^{a}, a halogenated lower alkyl group, (e.g., CF₃), a halogenated alkoxy group, an aryl group or a substituted aryl group.

Each R^{a} is independently —H, lower alkyl, substituted lower alkyl, aryl or substituted aryl.

Each R₂₃ is independently —H, lower alkyl or a phosphate protecting group. Preferably, R₂₃ is —H.

In another example, the phosphate transport inhibitor is a polymer comprising one or more phosphinyl phosphonate groups. Preferably, the phosphinyl phosphonate group is represented by Structural Formula (11), (12) or (13) (described in U.S. Application Publication No. 2004/0019020): 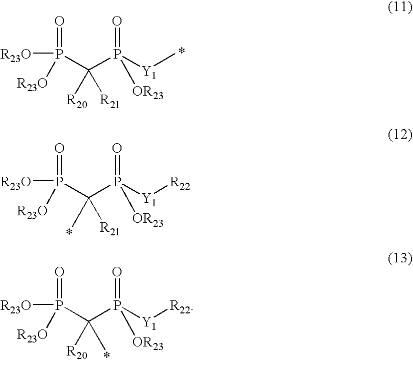
R₂₀, R₂₁, R₂₂, R₂₃ and Y₁ in Structural Formulas (11), (12) and (13) are as described above for Structural Formula (10). Also included are esters (e.g., alkyl esters) of the phosphinyl phosphonate group represented by Structural Formulas (11), (12) or (13).

Additional examples of phosphate transport inhibitors are compounds having a double bond or triple bond directly adjacent to a phosphinyl phosphate group, such as those represented by Structural Formulas (1 4), (15) or (16) (described in WO 2004/085448):

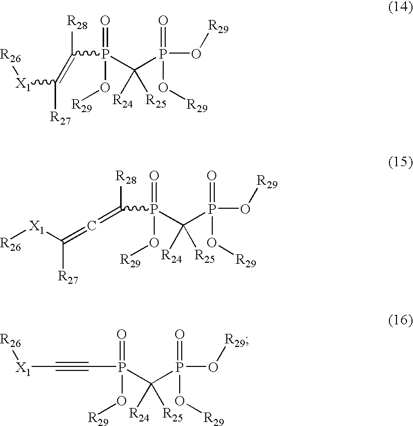

or a pharmaceutically acceptable salt or a prodrug thereof.

R₂₄ and R₂₅ are independently —H, —OR^{x}, —N(R^{x})₂, ═O, ═NR^{x}, an electron withdrawing group or a substituted or unsubstituted alkyl group.

R₂₆ is a substituted or unsubstituted hydrocarbyl group optionally interrupted by one or more nitrogen, oxygen or sulfur atoms.

R₂₇ and R₂₈ are independently —H, a halogen, a substituted or unsubstituted alkyl group, —C(O)R^{x}, —C(O)OR^{x}, —C(O)N(R^{x})₂, —OR^{x}, —SR^{x}, —S(O)OR^{x}, —S(O)OR^{x}, —S(O)N(R^{x})₂, —S(O)₂OR^{x}, —S(O)₂N(R^{x})₂, —N(R^{x)}₂, —N═N—R^{x}, —CN, or —NO₂.

Each R₂₉ is independently —H, a substituted or unsubstituted alkyl group, or a phosphate protecting group.

X₁ is a covalent bond, —CHY₂—, —C(Y₂)₂—, —C(O)—, —OC(O)—, —S—, —S(O)—, —S(O)₂—, —NR^{x}—, —O—, —C(S)—, —SC(O)—, —SC(S)—, —NHC(O)NH—, or —NHC(NH)NH—.

Y₂ is a halogen or lower alkyl group.

R^{x} is independently —H, a substituted or unsubstituted alkyl group, or a substituted or unsubstituted aryl group.

Preferably, when X₁ is a covalent bond, R₂₇ and R₂₈ are a halogen, an alkyl group substituted with an electron withdrawing group, —C(O)R^{x}, —C(O)OR^{x}, —C(O)N(R^{x})₂, —OR^{x}, —SR^{x}, —S(O)OR^{x}, —S(O)OR^{x}, —S(O)N(R^{x})₂, —S(O)₂OR^{x}, —S(O)₂N(R^{x})₂, —N(R^{x})₂, —N═N—R^{x}, —CN, or —NO₂.

Other phosphate transport inhibitors that can be used in the invention include polymers comprising one or more pendant alkenylene phosphinyl phosphonate or alkynylene phosphinyl phosphonate groups or pharmaceutically acceptable salts thereof or esters thereof. In one example, the alkenylene phosphinyl phosphonate or alkynylene phosphinyl phosphonate groups are represented by Structural Formula (17), (18), or (19) (described in WO 2004/085448): 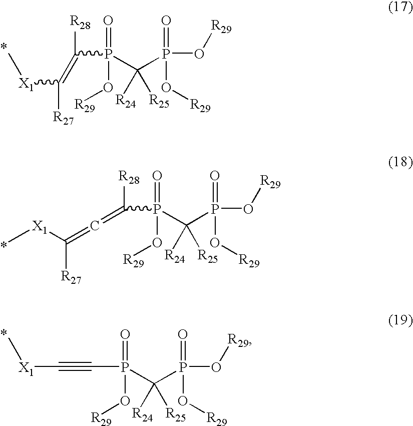
or a pharmaceutically acceptable salt thereof, where X₁, R₂₄, R₂₅, R₂₇, R₂₈ and R₂₉ are as defined above for Structural Formulas (14)-(16).

Suitable examples of HMG-CoA reductase inhibitors for the combination therapy of the invention include lovastatin (mevinolin) (e.g., Altocor® and Mevacor®) and related compounds; pravastatin (e.g., Pravachol®, Selektine®, and Lipostat®) and related compounds; simvastatin (e.g., Zocor®) and related compounds. Other HMG-CoA reductase inhibitors which can be employed in the present invention include fluvastatin (e.g., Lescol®); cerivastatin (e.g., Baycol® and Lipobay®); atorvastatin (e.g., Zarator® and Lipitor®); pitavastatin; rosuvastatin (visastatin) (e.g., Crestor®); quinoline analogs of mevalonolactone and derivatives thereof (see U.S. Pat. No. 5,753,675, the entire teachings of which are incorporated herein by reference); pyrazole analogs of mevalonolactone derivatives (see U.S. Pat. No. 4,613,610, the entire teachings of which are incorporated herein by reference); indene analogs of mevalonolactone derivatives (see WO 86/03488, the entire teachings of which are incorporated herein by reference); 6-[2-(substituted-pyrrol-1-yl)-alkyl)pyran-2-ones and derivatives thereof (see U.S. Pat. No. 4,647,576, the entire teachings of which are incorporated herein by reference); imidazole analogs of mevalonolactone (see WO 86/07054, the entire teachings of which are incorporated herein by reference); 3-hydroxy-4(dihydroxooxophosphorio)butanoic acid derivatives (see French Patent No. 2,596,393, the entire teachings of which are incorporated herein by reference); naphthyl analogs of mevalonolactone (see U.S. Pat. No. 4,686,237, the entire teachings of which are incorporated herein by reference); octahydronaphthalenes (see U.S. Pat. No. 4,499,289, the entire teachings of which are incorporated herein by reference); and quinoline and pyridine derivatives (see U.S. Pat. Nos. 5,506,219 and 5,691,322, the entire teachings of which are incorporated herein by reference). A statin, such as atorvastatin, fluvastatin, lovastatin, pravastatin, simvastatin, rosuvastatin, cerivastatin and pitavastatin, is preferred.

A large variety of organic and inorganic molecules are inhibitors to alkaline phosphatase (ALP) (see, for example, U.S. Pat. No. 5,948,630, the entire teachings of which are incorporated herein by reference). Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl)imidazo-[2,1-b]thiazole(napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

A “pharmaceutically acceptable salt” of a phosphate inhibitor represented by Structural Formulas (9)-(19) is as described above for a pharmaceutically acceptable salt of an amine polymer.

A “hydrocarbyl group” for Structural Formulas (9)-(19) is an aliphatic or arylene group, or a combination thereof, i.e., —(CH₂)_{z}— or —(CH₂)_{z}C₆H₄(CH₂)_{z}—, where z is a positive integer (e.g., from 1 to about 30), preferably between 6 and about 30, more preferably between 6 and about 15, and even more preferably between about 8 and about 14. The hydrocarbyl group may be optionally interrupted with one or more nitrogen, oxygen or sulfur atoms, or a combination thereof, or can have a backbone of only carbon atoms. Examples of hydrocarbyl groups include butylene, pentylene, hexylene, heptylene, octylene, nonylene, decylene, dodecylene, 4-oxaoctylene, 4-azaoctylene, 4-thiaoctylene, 3,6-dioxaoctylene, 3,6-diazaoctylene, and 4,9-dioxadodecane. Arylene groups can be interrupted by a nitrogen, oxygen or sulfur atom to form a heteroarylene group. An aliphatic hydrocarbyl group (or an aliphatic portion thereof) can optionally be saturated or can optionally have one or more double or triple bonds, which can be arranged to form a conjugated series of bonds. The conjugated bonds may or may not include the double or triple bond in the unsaturated phosphinyl phosphonate group.

An “aliphatic group” for Structural Formulas (9)-(19) is a straight chained, branched or cyclic non-aromatic hydrocarbon which is completely saturated or which contains one or more units of unsaturation. Typically, a straight chained or branched aliphatic group has from 1 to about 20 carbon atoms, preferably about 8 to about 14, and a cyclic aliphatic group has from 3 to about 10 carbon atoms, preferably from 3 to about 8. Examples of an aliphatic group include methyl, ethyl, n-propyl, iso-propyl, n-butyl, sec-butyl, tert-butyl, pentyl, hexyl, heptyl and octyl. An alkyl group is a completely saturated aliphatic group. A C1-C4 straight chained or branched alkyl group or a C3-C8 cyclic alkyl group is also referred to as a “lower alkyl” group.

The term “aryl group” for Structural Formulas (9)-(19) refers to carbocyclic aromatic groups such as phenyl, naphthyl, and anthracyl, and heteroaryl groups such as imidazolyl, isoimidazolyl, thienyl, furanyl, pyridyl, pyrimidyl, pyranyl, pyrazolyl, pyrrolyl, pyrazinyl, thiazoyl, isothiazolyl, oxazolyl, isooxazolyl, 1,2,3-trizaolyl, 1,2,4-triazolyl, and tetrazolyl. An “arylene group” is analogous to an aryl group, but is divalent instead of monovalent.

Heteroaryl groups for Structural Formulas (9)-(19) also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazolyl, benzoisothiazolyl, benzooxazolyl, benzoisooxazolyl, benzimidazolyl, quinolinyl, isoquinolinyl and isoindolyl.

An “olefin group” for Structural Formulas (9)-(19), as used herein, is a group containing a double bond. The double bond may be an E- or Z-isomer. Preferably the double bond is an E-isomer.

An “alkylene group” for Structural Formulas (9)-(19), as used herein, is represented by —(CH₂)ₙ—. n is an integer from 1-10, preferably 1-4.

In Structural Formulas (9)-(19), suitable substituents for a substituted aliphatic group, a substituted aryl group, a substituted hydrocarbyl group, a substituted alkyl group or a substituted heteroaryl group, are those which do not significantly lower the activity of the active compound or polymer (e.g., do not lower the activity by more than a factor of about two compared with the corresponding unsubstituted compound). Examples include -OH, a halogen (—Br, —Cl, —I and —F), —O(R_{y}), —O—CO—(R_{y}), —CN, —NO₂, —COOH, ═O, —NH₂ —NH(R_{y}), —N(R_{y})₂. —COO(R_{y}), —CONH₂, —CONH(R_{y}), —CON(R_{y})₂, —SH, —S(R_{y}), an aliphatic group, an aryl group and a heteroaryl group. Each R^{b} is independently —H, an alkyl group or an aryl group. A substituted aliphatic group, a substituted aryl group a substituted hydrocarbyl group, a substituted alkyl group or a substituted heteroaryl group, can have more than one substituent.

The term “electron withdrawing group” for Structural Formulas (9)-(19), as it is used herein, has the meaning commonly afforded the term in the art. Specifically, an electron withdrawing group is a substituent which results in a phenyl ring having less electron density when the group is present on the phenyl ring than when it is absent. Electron withdrawing groups have a Hammet sigma value greater than zero (see, for example, C. Hansch, A. Leo and D. Hoeckman, “Exploring QSAR Hydrophobic, Electronic and Steric Constants”, American Chemical Society (1995), pages 217-32, the contents of which are incorporated herein by reference). Examples of electron withdrawing groups include halogens, —NO₂, —CN, —CF₃ and —OCF₃. A halogen, particularly fluoride, is a preferred electron withdrawing group.

“Phosphate protecting groups” for Structural Formulas (9)-(19) are groups that are generally removed from both the phosphinyl and phosphonate moieties by hydrolysis, thereby obtaining the free acid or salt form of the phosphinyl phosphonate. Phosphate protecting groups can be chosen so that they are removed at a desired rate or under desired conditions. One example of a phosphate protecting group is an ester of a simple alcohol (e.g., ethanol), diols or polyols (e.g., sugars such as glucose) or polymeric alcohols (e.g., polyvinyl alcohol, polyallyl alcohol, polysaccharides). Phosphate protecting groups also include amines that are capable of forming a phosphorus-nitrogen bond. Another type of phosphate protecting group is an acid halide or other activated carboxylic acid, which can form an acid anhydride with the phosphinyl phosphonate group. A phosphate protecting group can also be a polymer, which has pendant groups capable of forming a hydrolyzable bond with the phosphinyl phosphonate group. Suitable pendant groups include hydroxyl groups, amine groups and carboxylic acids and derivatives (e.g., acid halides, acid anhydrides, etc.) thereof.

The pharmaceutical compositions of the invention optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. In some embodiments, the pharmaceutically acceptable carrier, diluent or excipient does not include a zinc salt.

The carriers, diluents and excipients are “acceptable” in the sense of being compatible with the other ingredients of the pharmaceutical composition and not deleterious to the recipient thereof. The pharmaceutical compositions can conveniently be presented in unit dosage form and can be prepared by any suitable method known to the skilled artisan. In general, the pharmaceutical compositions are prepared by uniformly and intimately bringing into association the pharmaceutically acceptable zinc salt with the carriers, diluents and/or excipients and then, if necessary, dividing the product into unit dosages thereof.

The pharmaceutical compositions of the invention can be formulated as a tablet, sachet, slurry, food formulation, troche, capsule, elixir, suspension, syrup, wafer, chewing gum or lozenge.

A syrup formulation generally consists of a suspension or solution of the phosphate binding polymer or salt in a liquid carrier, for example, ethanol, glycerine or water, with a flavoring or coloring agent.

Where the pharmaceutical compositions are in the form of a tablet, one or more pharmaceutical carriers routinely used for preparing solid formulations can be employed. Examples of such carriers include magnesium stearate, starch, lactose and sucrose. For example, tablet formualtions for oral use can be obtained by combining the active compound with a one or more excipients, optionally grinding a resulting mixture, and processing the mixture of granules, after adding suitable auxiliaries, if desired, to obtain tablet cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Where the pharmaceutical compositions are in the form of a capsule, the use of routine encapsulation is generally suitable, for example, using the aforementioned carriers in a hard gelatin capsule shell. For example, methods for encapsulating compositions (such as in a coating of hard gelatin or cyclodextran) are known in the art (Baker, et al., “Controlled Release of Biological Active Agents”, John Wiley and Sons, 1986, the entire teachings of which are incorporated herein by reference). Where the pharmaceutical compositions are in the form of a soft gelatin shell capsule, carriers routinely used for preparing dispersions or suspensions can be considered, for example, aqueous gums, celluloses, silicates or oils, and are incorporated in a soft gelatin capsule shell. The pharmaceutical compositions can also be in the form of a push-fit capsule made of a suitable material, such as gelatin, as well as soft, sealed capsule made of a suitable material, for example, gelatin, and a plasticizer, such as glycerol or sorbitol. The push-fit capsules can contain the pharmaceutically acceptable zinc salt in admixture with filler such as lactose, binders such as starches, and/or lubricants, such as talc, and, optionally, stabilizers. In soft capsules, the pharmaceutically acceptable zinc salt can be dissolved or suspended in suitable liquids, such as fatty oils, liquid paraffin, or liquid polyethylene glycols. In addition, stabilizers can optionally be added.

In a preferred embodiment, the pharmaceutical compositions of the invention are formulated as a tablet.

In another preferred embodiment, the pharmaceutical compositions of the invention are formulated as a powder formulation which can be easily packaged as a sachet or a tub from which a unit dose is measured by, e.g., a spoon or cup, or an instrument capable of dispensing a pre-defined dosage amount. The powder formulation preferably further includes a pharmaceutically acceptable anionic polymer, such as alginate (e.g., sodium alginate, potassium alginate, calcium alginate, magnesium alginate, ammonium alginate, esters of alginate, etc.), carboxymethyl cellulose, poly lactic acid, poly glutamic acid, pectin, xanthan, carrageenan, furcellaran, gum arabic, karaya gum, gum ghatti, gum carob and gum tragacanth (see U.S. Provisional Application No. 60/717,200 filed on Sep. 15, 2005, the entire teachings of which are incorporated herein by reference). One or more sweeteners and/or flavorants can be optionally included in the powder formulation.

Though the above description is directed toward routes of oral administration of pharmaceutical compositions consistent with embodiments of the invention, it is understood by those skilled in the art that other modes of administration using vehicles or carriers conventionally employed and which are inert with respect to the pharmaceutically acceptable zinc salts may be utilized for preparing and administering the pharmaceutical compositions. Illustrative of such methods, vehicles and carriers are those described, for example, in Remington's Pharmaceutical Sciences, 18^{th} ed. (1990), the disclosure of which is incorporated herein by reference.

A “subject” is preferably a human, but can also be another animal in need of treatment with a pharmaceutically acceptable zinc salt as described above, e.g., companion animals (e.g., dogs, cats, and the like), farm animals (e.g., cows, pigs, horses and the like) and laboratory animals (e.g., rats, mice, guinea pigs and the like). As used herein, a subject in need of treatment with a pharmaceutically acceptable zinc salt include subjects with diseases and/or conditions that can be treated with a pharmaceutically acceptable zinc salt to achieve a beneficial therapeutic and/or prophylactic result. A beneficial outcome includes a decrease in the severity of symptoms or delay in the onset of symptoms, increased longevity and/or more rapid or more complete resolution of the disease or condition. For example, a subject in need of treatment typically has elevated serum phosphate levels, hyperphosphatemia, resulting from, for example, impaired kidney function or hypoparathyroidism. A subject in need of treatment also includes a subject with chronic renal failure. Other examples of subjects in need of treatment with a pharmaceutically acceptable zinc salt include patients with a disease associated with disorders of phosphate metabolism. Examples of diseases and/or disorders of this type include hyperparathyroidism, inadequate renal function, and hyperphosphatemia.

“Treating,” as used herein includes both therapeutic and prophylactic treatments.

An “effective amount” of a pharmaceutically acceptable zinc salt or a mixture of pharmaceutically acceptable zinc salts is a quantity that results in a beneficial clinical outcome of the condition being treated with the salt(s) compared with the absence of treatment. An “effective amount” of a pharmaceutically acceptable zinc salt or a mixture of pharmaceutically acceptable zinc salts is also an amount which achieves a beneficial propylactic effect when given to a subject at risk of development of, for example, renal failure, hypoparathyroidism, or hyperphosphatemia, to prevent onset of these diseases and/or conditions. The amount of a pharmaceutically acceptable zinc salt or a mixture of pharmaceutically acceptable zinc salts administered to the subject will depend on the degree, severity, and type of the disease or condition, the amount of therapy desired, and the release characteristics of the pharmaceutical formulation. It will also depend on the subject's health, size, weight, age, sex and tolerance to drugs. Typically, the compositions of the invention are administered for a sufficient period of time to achieve the desired therapeutic effect. Typically between about 0.1 mg per day and about 10 g per day of the pharmaceutically acceptable zinc salt or a mixture of pharmaceutically acceptable zinc salts (alternatively between about 1 mg per day and about 5 g per day, alternatively between about 0.5 g per day and about 5 g per day, alternatively between about 0.5 per day and about 3 g per day) is administered to the subject in need of treatment. These dosages can be administered several times/day (e.g., 2, 3, 4 or 5 times/day) or once/day.

An effective amount of a phosphate sequestrant as described above, such as an amine polymer (e.g., sevelamer, colesevelam and colestipol), or a pharmaceutically acceptable lanthanum (e.g., Fosrenol®) or calcium (PhosLo®) salt, is generally known in the art. Also, an effective amount of a phosphate transport inhibitor, an HMG-CoA reductase inhibitor or an alkaline phosphatase inhibitor is generally known in the art. When one or more of these second agents, e.g., a phosphate sequestrant, a phosphate transport inhibitor, an HMG-CoA reductase inhibitor and an alkaline phosphatase inhibitor, are used in combination with the pharmaceutically acceptable zinc salt, the effective amount of the pharmaceutically acceptable zinc salt is adjusted to take into account the effective amount of the second agent(s) to achieve the desired phosphate binding capacity.

For example, when a pharmaceutical composition of the invention comprises a pharmaceutically acceptable zinc salt as described above and an amine polymer (preferably an aliphatic amine polymer), typically between about 5 mg per day and about 15 g per day of the pharmaceutical composition (alternatively between about 50 mg per day and about 12 g per day, alternatively between about 0.5 g per day and about 12 g per day, alternatively between about I g per day and about 12 g per day, alternatively between about 0.5 g per day and about 10 g per day, alternatively between about 1 g per day and about 10 g per day, alternatively between about 2 g per day and about 10 g, alternatively between about 3 g per day and about 10 g per day, alternatively between about 1 g per day and about 8 g per day, alternatively between about 2 g per day and about 8 g per day, alternatively between about 2 g per day and about 6 g per day, alternatively between about 2 g per day and about 5 g per day) is administered to the subject in need of treatment. Frequency of administration is as described above when the pharmaceutically acceptable zinc salt is administered. In one specific example, about 0.8-7.2 g (e.g., 1.2 g, 1.6 g, 1.8 g, 2.0, 2.4 g, 3.0 g, 3.2 g, 3.6 g, 4.0 g or 4.8 g per dose for 2-3 times per day, or 3.0 g, 3.2 g, 3.6 g, 4.0 g or 4.8 g, 5.4 g, 6.0 g, 6.2 g, 6.6g, 7.0 g or 7.2 g per dose for once per day) of the pharmaceutical composition is administered per day. The pharmaceutical compositions can be administered at least four times per day with meals, at least three times per day with meals, at least twice per day with meals, at least once per day with meals, (see US Provisional Application No. 60/623,985, “Once a day formulation for phosphate binders” filed Nov. 1, 2004, the entire contents of which are incorporated herein by reference).

Typically, the composition of the invention can be administered before or after a meal, or with a meal. Preferably, the effective amount of the composition of the invention is administered several times/day or once/day with a meal. As used herein, “before” or “after” a meal is typically within two hours, preferably within one hour, more preferably within thirty minutes, most preferably within ten minutes of commencing or finishing a meal, respectively.

The pharmaceutically acceptable zinc salt(s) can be administered as multiple dosage units or preferably as a single dosage unit. As used herein a dosage unit may be a tablet, sachet, slurry, food formulation, troche, capsule, elixir, suspension, syrup, wafer, chewing gum or the like prepared by art recognized procedures. Preferably a dosage unit is a tablet, capsule, sachet, slurry, suspension or food formulation, more preferably the dosage unit is a tablet, slurry, suspension or food formulation, most preferably the dosage unit is a tablet or sachet. Typically, the desired dose of a pharmaceutically acceptable zinc salt(s) is administered as multiple tablets or capsules, or a single dose of a sachet, slurry, food formulation, suspension or syrup.

Those skilled in the art will be aware that the amounts of the various components of the pharmaceutical compositions of the invention to be administered in accordance with the method of the invention to a subject will depend upon those factors noted above.

The invention is illustrated by the following examples which are not intended to be limiting in any way.

### EXEMPLIFICATION

### Example 1A

### Mixture of Sevelamer.HCl and Zinc Salt: Zinc Acetate Dihydrate

The pH of a stirred mixture of sevelamer hydrochloride (10 g) in deionized water (500 mL) was adjusted to pH 9-10 with the addition of 50% aqueous NaOH. A solution of zinc acetate dihydrate (38.5 g) in deionized water (200 mL) was then added to the sevelamer hydrochloride mixture with stirring. After stirring at room temperature for 1 hour, the pH of the mixture was pH 7. The mixture was then filtered, and the collected solids were lyophilized to afford 24 g. Anal. Found: C, 33.85; N, 8.0; Zn, 17.18.

### Example 1B

### Mixture of Sevelamer.HCl and Zinc Salt: Zinc Acetate Dihydrate

The pH of a stirred mixture of sevelamer hydrochloride (10 g) in deionized water (500 mL) was adjusted to pH 9-10 with the addition of 50% aqueous NaOH. A solution of zinc acetate dihydrate (38.5 g) in deionized water (200 mL) was then added to the sevelamer mixture with stirring. After stirring at room temperature for 1 hour, the pH of the mixture was pH 7. The mixture was then lyophilized without filtration to afford 43 g. Anal. Found: C, 31.26; N, 5.21; Zn, 28.30.

### Example 2

### Mixture of Sevelamer.HCl and Zinc Salt: Zinc Chloride

The pH of a stirred mixture of sevelamer.HCl (10 g) in deionized water (400 mL) was adjusted to pH 9-10 with the addition of 50% aqueous NaOH. A solution of zinc chloride (23.9 g) in deionized water (100 mL) was then added to the sevelamer hydrochloride mixture with stirring. After stirring at room temperature for 1 hour, the mixture was lyophilized to afford 20.64 g. Anal. Found: C, 25.33; N, 8.75; Zn, 18.68.

### Example 3

### Mixture of Sevelamer.HCl and Zinc Salt: Zinc Oxide

To a stirred suspension of sevelamer hydrochloride (10 g), ZnO (14.24 g), and deionized water (500 mL) was added 50% aqueous NaOH (5 g) until the stirred mixture had pH 13. After stirring at room temperature, the mixture was filtered. The solid material was washed on the funnel with deionized water and lyophilized to afford 21.45 g. Anal. Found: C, 22.17; H, 4.23; N, 7.57; Zn, 65.47.

### Example 4

### Mixture of Sevelamer.HCl and Zinc Salt: Zinc Oxide

To a mixture of ZnO (28.48 g) in ammonium hydroxide (1 L, 28-30% aqueous solution) was added sevelamer hydrochloride (10 g). After stirring 48 hours, the mixture was filtered. The material collected was washed with deionized water on the funnel and lyophilized to afford 27.5 g. Anal. Found: C, 17.84; H, 3.21; N, 5.96; Zn, 77.25.

### Example 5

### Admixture of sevelamer.HCl—ZnO (1:1 by weight)

In a 60 mL Nalgene bottle was added zinc oxide (3.00 g, nanopowder) and sevelamer hydrochloride (3.00 g). The bottle was shaken by hand for about 5 minutes to mix.

### Example 6

### Admixture of sevelamer.HCl—ZnO (2:1 by weight)

In a 60 mL Nalgene bottle was added zinc oxide (3.00 g, nanopowder) and sevelamer hydrochloride (6.00 g). The bottle was shaken by hand for about 5 minutes to mix.

### Example 7

### Mixture of Poly(acrylic acid) and ZnCl₂

To a solution of poly(acrylic acid, sodium salt) (50 g of a 40 wt. % aqueous solution) in deionized water (100 mL) was added a solution of zinc chloride (30 g) in deionized water (50 mL). After stirring overnight, the mixture was filtered, washed with water (100 mL), and dried in a forced-air oven at 60° C. to afford 25.9 g of a white solid.

### Example 8

### Poly(zinc acrylate)

To a solution of zinc acrylate (20 g) in deionized water (380 mL) under a nitrogen atmosphere was added 2,2′-azobisamidinopropane dihydrochloride (0.2 g dissolved in 1 mL water) and the solution was heated to 60° C. overnight. The reaction mixture was filtered and the solid was washed with deionized water (2×1.5 L) to afford 20.52 g after drying in a forced-air oven at 60° C.

### Example 9

### Crosslinked Polydiallylamine (PDA) Polymer in the Presence of Zinc Salt: 20 g PDA.HCl, 15% ZnO (on the basis of the weight of PDA.HCl). 18 mol % epichlorohydrin (on the basis of the molecular weight of a repeat unit of PDA)

Partially neutralized poly(diallylamine)hydrochloride: Diallylamine (2000.3 g) was added slowly over a period of 2 days to concentrated HCl (2035.6 g). The temperature of the reaction was maintained below 10° C. by cooling the flask in an ice-salt-water bath, and by adjusting the addition rate. The room temperature pH of the resulting diallylamine hydrochloride solution (68.16% diallylamine hydrochloride) was 0.005. To a 12 L 4-neck round bottom flask equipped with an overhead stirrer and an air condenser, was added diallylamine hydrochloride (3667.8 g of a 68.16% solution) and deionized water (4665.5 g). The resulting solution had pH 0.741. To the flask was added NaOH (66.8 g of a 50% aqueous solution). The resulting solution had pH 2.554. Nitrogen gas was bubbled through the solution, via a stainless steel needle, with stirring, and venting on top of an air condenser for 2 hours. The nitrogen line was put on top of the air condenser with positive pressure from a mineral oil bubbler. To the flask was added 125.0 g of freshly made 20% 2,2′-azobis(2-amidinopropane)dihydrochloride in deionized water. This was added via syringe through-a septum. The 2,2′-azobis(2-amidinopropane)dihydrochloride solution was not degassed with nitrogen. The solution was heated to 60° C. over a period of 1 hour and 8 minutes with a heating mantle connected to a J-Kem temperature controller. The solution was then heated at 60° C. for 18 hours. After cooling to room temperature, the solution was a dark orange viscous, flow able, clear solution. The flask contents were then combined with deionized water (4166.7 g). The resulting solution had pH 4.414. SEC (size exclusion chromatography) analysis: Mw 61494 Daltons; Polydispersity 2.43. To a solution of 900 g poly(diallylamine)hydrochloride (28.6% (w/w) aqueous solution) was added 49.95 mL 50% aqueous NaOH. The solution had pH 10.13 and was equivalent to 26.3%(w/w) poly(diallylamine)hydrochloride.

Crosslinked polydiallylamine (PDA) hydrochloride in the presence of zinc oxide: To a partially neutralized solution of PDA (76 g) prepared as described above was added zinc oxide (3.05 g). After stirring for 20 minutes at room temperature, epichlorohydrin (2.11 mL) was added. A gel formed within 20 minutes. After curing at room temperature overnight, the gel was broken into small pieces and suspended into deionized water (4 L). After stirring for 20 minutes, the suspension was filtered. The filtered polymer was suspended again in deionized water (4 L), stirred for 20 minutes, and filtered. The filtered polymer was lyophilized to afford 21.44 g. Anal. Found: C, 48.54; H, 8.41; N, 8.60; Zn, 11.46.

### Example 10

### Crosslinked Polyethylenimine (PEI) Polymer in the Presence of Zinc Salt

To a stirred mixture of polyethylenimine (40.0 g, low MW), deionized water (60 mL) and zinc oxide (9.36 g) was added epichlorohydrin (3.24 mL). After stirring overnight, another portion of epichlorohydrin (3.24 mL) was added. A gel formed and was allowed to cure at room temperature overnight. The gel was broken into small pieces, washed with deionized water (2×4 L), and dried in a forced-air oven at 60° C. to afford 33.68 g.

### Example 11

### Crosslinked Polyethylenimine (PEI) Polymer in the Presence of Zinc Salt: 20 g PEI, 43% ZnO (on the basis of the weight of PEI), 9 mol % epichlorohydrin (on the basis of the molecular weight of a repeat unit of PEI)

To a solution of polyethylenimine (20 g, MW 25000, water free) in deionized water (80 g) was added ZnO (9.36 g). After stirring for 20 minutes at room temperature, epichlorohydrin (3.24 mL) was added. After curing at room temperature overnight the gel was broken into small pieces and suspended into deionized water (4 L). After stirring for 20 minutes, the suspension was filtered. The filtered polymer was suspended again in deionized water (4 L), stirred for 20 minutes, and filtered. The filtered polymer was dried in a forced-air oven at 60° C. to afford 30.97 g. Anal. Found: C, 37.30; H, 7.64; N, 18.36; Zn, 23.99.

### Example 12A

### Crosslinked Polyallylamine (PAA) Polymer in the Presence of Zinc Salt: Zinc Oxide

To a solution of polyallylamine hydrochloride (700 g of 50% (w/w) aqueous solution) was added deionized water (1050 g) followed by NaOH (185.38 g of 50% (w/w) NaOH in water). This solution contains the equivalent of 18.08% (w/w) polyallylamine hydrochloride.

To a partially neutralized poly(allylamine)hydrochloride solution (55.3 g) prepared as described above was added 3.62 g of 50% aqueous NaOH (to pH 14), followed by ZnO (14.24 g, nanopowder) then epichlorohydrin (0.97 g). A gel formed. After curing at room temperature, the gel was broken into small pieces, washed with deionized water (2×1 L), lyophilized, ground in a coffee mill, and sieved through an 80 mesh sieve to afford 17.5 g. Anal. Found: C, 19.08; H, 3.97; N, 6.62; Zn, 68.33.

### Example 12B

### Crosslinked Polyallylamine (PAA) Polymer in the Presence of Zinc Salt: 10 g PAA.HCl, 142% ZnO (on the basis of the weight of PAA.HCl) 9.8 mol % epichlorohydrin (on the basis of the molecular weight of a repeat unit of PAA)

To a partially neutralized poly(allylamine)hydrochloride solution (55.3 g) prepared as described in Example 12 A was added 3.62 g of 50% aqueous NaOH (to pH 14), followed by ZnO (14.24 g, nanopowder) then epichlorohydrin (0.97 g). A gel formed. After curing at room temperature, the gel was broken into small pieces, washed with deionized water (3×1 L), and lyophilized to afford 22.1 g. Anal. Found: C, 18.69; H, 3.53; N, 6.13; Zn, 46.89.

### Example 13

### Crosslinked Polyallylamine (PAA) Polymer in the Presence of Zinc Salt: 10 g PAA.HCl 71% ZnO (on the basis of the weight of PAA.HCl), 9.8 mol % epichlorohydrin (on the basis of the molecular weight of a repeat unit of polyallylamine)

To a partially neutralized poly(allylamine)hydrochloride solution (55.3 g) (see Example 12A) was added 3.6 g of 50% aqueous NaOH (to pH 14), followed by ZnO (7.12 g, nanopowder) then epichlorohydrin (0.97 g). A gel formed. After curing at room temperature, the gel was broken into small pieces, washed with deionized water (3×1 L), and lyophilized to afford 13.1 g. Anal. Found: C, 27.26; H, 5.52; N, 9.11; Zn, 47.79.

### Example 14

### Crosslinked Polyallylamine (PAA) Polymer in the Presence of Zinc Salt: 10 g PAA.HCl, 36% ZnO (on the basis of the weight of PAA.HCl). 9.8 mol % epichlorohydrin (on the basis of the molecular weight of a repeat unit of PAA)

To a partially neutralized poly(allylamine)hydrochloride solution (55.3 g) (see Example 12A) was added 3.74 g of 50% aqueous NaOH (to pH 14), followed by ZnO (3.56 g, nanopowder) then epichlorohydrin (0.97 g). A gel formed. After curing at room temperature, the gel was broken into small pieces, washed with deionized water (3×1 L), and lyophilized to afford 9.9 g. Anal. Found: C, 36.73; H, 7.30; N, 12.38; Zn, 27.87.

### Example 15

### Crosslinked Polyallylamine (PAA) Polymer in the Presence of Zinc Salt: 10 g PAA.HCl 36% ZnO (on the basis of the weight of PAA.HCl) 20 mol % epichlorohydrin (on the basis of the molecular weight of a repeat unit of PAA)

To a partially neutralized poly(allylamine)hydrochloride solution (55.3 g) (see Example 12A) was added 3.36 g of 50% aqueous NaOH (to pH 14), followed by ZnO (3.56 g, nanopowder) then epichlorohydrin (1.94 g). A gel formed. After curing at room temperature, the gel was broken into small pieces, washed with deionized water (3×0.75 L), and lyophilized to afford 11.4 g. Anal. Found: C, 37.16; H, 7.34; N, 11.72; Zn, 33.31.

### Example 16

### Crosslinked Polyallylamine (PAA) Polymer in the Presence of Zinc Salt: 10 g PAA.HCl, 36% ZnO (on the basis of the weight of PAA.HCl), 39 mol % epichlorohydrin (on the basis of the molecular weight of a repeat unit of PAA)

To a partially neutralized poly(allylamine)hydrochloride solution (55.3 g) (see Example 12A) was added 3.2 g of 50% aqueous NaOH (to pH 14), followed by ZnO (3.56 g, nanopowder) then epichlorohydrin (3.88 g). A gel formed. After curing at room temperature, the gel was broken into small pieces, washed with deionized water (3×0.75 L), and lyophilized to afford 11.4 g. Anal. Found: C, 37.5; H, 7.18; N, 10.00; Zn, 25.44.

### Example 17

### Crosslinked Polyallylamine (PAA) Polymer in the Presence of Zinc Salt: 10 g PAA.HCl, 14% ZnO (on the basis of the weight of PAA.HCl), 9.8 mol % epichlorohydrin (on the basis of the molecular weight of a repeat unit of PAA)

To a partially neutralized poly(allylamine)hydrochloride solution (55.3 g) (see Example 12A) was added 3.1 g of 50% aqueous NaOH (to pH 14), followed by ZnO (1.42 g, nanopowder). After stirring for 30 minutes, epichlorohydrin (0.97 g) was added. A gel formed. After curing at room temperature, the gel was broken into small pieces, washed with deionized water (3×1 L), and lyophilized to afford 14 g. Anal. Found: C, 43.81; H, 9.36; N, 15.09; Zn, 16.49.

### Example 18

### Crosslinked Polyallylamine (PAA) Polymer in the Presence of Zinc Salt: 10 g PAA.HCl, 7% ZnO (on the basis of the weight of PAA.HCl) 9.8 mol % epichlorohydrin (on the basis of the molecular weight of a repeat unit of PAA)

To a partially neutralized poly(allylamine)hydrochloride solution (55.3 g) (see Example 12A) was added 3.31 g of 50% aqueous NaOH (to pH 14), followed by ZnO (0.71 g, nanopowder), and epichlorohydrin (0.97 g). A gel formed. After curing at room temperature, the gel was broken into small pieces, washed with deionized water (3×1 L), and lyophilized to afford 14 g. Anal. Found: C, 48.39; H, 10.19; N, 16.60; Zn, 8.66.

### Example 19

### Crosslinked Polyallylamine (PAA) Polymer in the Presence of Zinc and Magnesium Compounds (1:1 by weight)

To a partially neutralized poly(allylamine)hydrochloride solution (110.6 g) (see Example 12A) was added ZnO (3.53 g, nanopowder) and MgO (3.53, −325 mesh) was added. After stirring for 1 hour at room temperature, epichlorohydrin (1.94 g) was added. A gel formed. After curing at room temperature, the gel was broken into small pieces, and washed with deionized water (4×2 L). The washed gel was split into two portions. One portion was lyophilized to give 10.64 g (Example 19-#1). Anal. Found: C, 37.58; H, 8.12; N, 12.45; Cl, 6.06; Mg, 5.42; Zn, 15.73. The other portion was dried at 60° C. in a forced-air oven to give 11.17 g (Example 19-#2). Anal. Found: C, 38.50; H, 8.00; N, 13.05; Cl, 6.33; Mg, 8.54; Zn, 16.69.

### Example 20

### Crosslinked Polyallylamine (PAA) Polymer in the Presence of Zinc and Magnesium Compounds (1:4 by weight)

To a partially neutralized poly(allylamine)hydrochloride solution (110.6 g) (see Example 12A) was added ZnO (1.76 g, nanopowder) and MgO (7.06, -325 mesh) was added. After stirring for 1 hour at room temperature, epichlorohydrin (1.94 g) was added. A gel formed. After curing at room temperature, the gel was broken into small pieces, and washed with deionized water (4×2 L). The washed gel was dried at 60° C. in a forced-air oven to give 25.9 g. Anal. Found: C, 32.01; H, 7.65; N, 10.81; Cl, 6.07; Mg, 8.88; Zn, 4.02.

### Example 21

### Effects of Zinc Compounds for Reducing Urinary Phosphate Levels

House male Sprague Dawley (SD) rats were used for the experiments. The rats were placed singly in wire-bottom cages, fed with Purina 5002 diet, and allowed to acclimate for at least 5 days prior to experimental use.

To establish baseline phosphorus excretion, the rats were placed in metabolic cages for 48 hours. Their urine was collected and its phosphorus content analyzed with a Hitachi analyzer to determine phosphorus excretion in mg/day. Any rats with outlying values were excluded; and the remainder of the rats was distributed into groups.

Purina 5002 was used as the standard diet. The pharmaceutically acceptable zinc salt being tested was mixed with Purina 5002 to result in a final concentration as indicated in Table 1 by weight. Cellulose at 0.5% by weight was used as a negative control. For each rat, 200 g of diet was prepared.

Each rat was weighed and placed on the standard diet. After 4 days the standard diet was replaced with the treatment diet (or control diet for the control group). On days 5 and 6, urine samples from the rats at 24 hours (±30 minutes) were collected and analyzed. The test rats were again weighed, and any weight loss or gain was calculated. Any remaining food was also weighed to calculate the amount of food consumed per day. A change in phosphorus excretion relative to baseline and cellulose negative control was calculated using Excel program. A summary of comparison of the amounts of urinary phosphate obtained from the test rats is shown in Table 1.

TABLE 1
Zinc Compounds: In Vivo Urinary Phosphate Excretion

| | | Phosphate | % Urinary Phosphate | |
|---|---|---|---|---|
| | | Excretion | Relative to that | |
| | Zinc Compound | (mg/day) | of Control Animals | % of Diet |
| 1 | Zinc citrate | 12 | 70.8 | 0.5 |
| | dihydrate | | | |
| 2 | Zinc glycinate | 15.5 | 79.7 | 0.5 |
| | monohydrate | | | |
| 3 | Zinc acetate | 9.4 | 56.7 | 0.5 |
| | dehydrate | | | |
| 4 | Zinc chloride | 8.8 | 50.6 | 0.5 |
| 5 | Zinc oxide | 3.2 | 18.4 | 0.5 |
| 6 | Zinc Oxalate | 16.2 | 78.0 | 0.5 |
| 7 | Zinc Formate | 10.1 | 48.5 | 0.5 |
| 8 | zinc oxide, | 9.7 | 50.7 | 0.25 |
| | nanopowder | | | |

As shown in Table 1, the amounts of urinary phosphate obtained from the rats which went through the zinc therapy with zinc oxide, zinc chloride, zinc acetate dehydrate, zinc glycinate monohydrate, zinc citrate dihydrate, zinc oxalate and zinc formate are much lower than those of a control which did not go through the zinc therapy.

### Example 22

### Effects of Compositions Comprising a Zinc Compound for Reducing Urinary Phosphate Levels

Effects of various compositions comprising a pharmaceutically acceptable zinc salt on reducing urinary phosphate levels were studied using house male Sprague Dawley (SD) rats as described in Example 21. A change in phosphorus excretion relative to baseline and cellulose negative control was calculated using Excel program. A summary of comparison of the amounts of urinary phosphate obtained from the test rats is shown in the Table 2 below.

TABLE 2
Amounts of Urinary Phosphate in Tested SD Rats

| | % Urinary Phosphate | |
|---|---|---|
| | Relative to that | |
| Treatment | of Control Animals | % of Diet |
| Example 7 | 88.9 | 0.5 |
| Example 1A | 65.2 | 0.5 |
| Example 1B | 39.5 | 0.5 |
| Example 5 | 64.8 | 0.25 |
| Example 6 | 71.9 | 0.25 |
| Example 3 | 27.8 | 0.5 |
| Example 12A | 19.1 | 0.5 |
| Example 12A | 63.8 | 0.25 |
| Example 14 | 23.0 | 0.5 |
| Example 12B | 54.0 | 0.25 |
| Example 14 | 51.6 | 0.25 |
| Example 15 | 55.9 | 0.25 |
| Example 17 | 57.8 | 0.25 |
| Example 18 | 69.5 | 0.25 |
| Example 11 | 71.7 | 0.25 |
| Example 9 | 84.2 | 0.25 |
| Example 19 | 78.9 | 0.25 |
| Example 20 | 64.0 | 0.25 |

As shown in Table 2, the amounts of urinary phosphate obtained from the rats which went through the zinc therapy with the listed various compositions comprising a S pharmaceutically acceptable zinc salt are much lower than those of a control which did not go through the zinc therapy.

While this invention has been particularly shown and described with references to preferred embodiments thereof, it will be understood by those skilled in the art that various 10 changes in form and details may be made therein without departing from the scope of the invention encompassed by the appended claims.

1. A method of treating hyperphosphatemia in a subject, comprising the step of administering to the subject an effective amount of a pharmaceutically acceptable zinc salt, wherein the pharmaceutically acceptable zinc salt is administered in an amount of between 0.1 mg/day and 10 g/day.

2. (canceled)

3. (canceled

4. The method of claim 1, wherein the zinc salt is selected from the group consisting of zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine, zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate).

5. (canceled)

6. (canceled)

7. The method of claim 4, wherein the zinc salt is zinc oxide.

8. The method of claim 1, wherein the method further comprises co-administering a phosphate sequestrant to the subject.

9. The method of claim 8, wherein the phosphate sequestrant is selected from the group consisting of a pharmaceutically acceptable calcium salt, a pharmaceutically acceptable aluminum salt, a pharmaceutically acceptable magnesium salt, a pharmaceutically acceptable lanthanum salt and a pharmaceutically acceptable iron salt.

10. The method of claim 8, wherein the phosphate sequestrant is an aliphatic amine polymer.

11. (canceled)

12. The method of claim 10, wherein the phosphate sequestrant is an aliphatic amine polymer comprising one or more repeat units represented by a formula selected from the group consisting of: 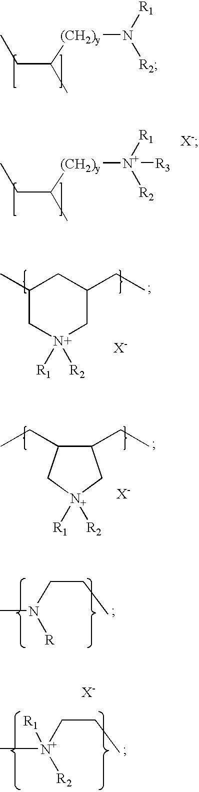
or a salt or a copolymer thereof, wherein:
y is zero or an integer from one to ten;
R, R₁, R₂ and R₃, independently, is H, a substituted or unsubstituted alkyl group or an aryl group; and
X⁻ is an exchangeable negatively charged counterion.

13. (canceled)

14. The method of claim 12, wherein the aliphatic amine polymer is a polyallylamine cross-linked by means of a multifunctional cross-linking agent.

15. The method of claim 14, wherein the polyallylamine is sevelamer.

16. (canceled)

17. (canceled)

18. (canceled)

19. (canceled)

20. (canceled)

21. (canceled)

22. (canceled)

23. (canceled)

24. (canceled)

25. A pharmaceutical composition, comprising:
a) a pharmaceutically acceptable carrier;
b) a pharmaceutically acceptable zinc salt; and
c) an amine polymer,
wherein the molar ratio of the zinc ion of the zinc salt to amine nitrogen atoms in the amine polymer is 0.1-3.0.

26. The pharmaceutical composition of claim 25, provided that the pharmaceutical composition does not comprise a zinc salt of a fatty acid.

27. The pharmaceutical composition of claim 25, wherein the zinc salt is selected from the group consisting of zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine, zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate).

28. The pharmaceutical composition of claim 27, wherein the composition comprises a mixture of zinc salts.

29. The pharmaceutical composition of claim 27, wherein the zinc salt is selected from the group consisting of zinc oxide, zinc acetate, zinc citrate, zinc carbonate and zinc tartrate.

30. The pharmaceutical composition of claim 29, wherein the zinc salt is zinc oxide.

31. The pharmaceutical composition of claim 25, wherein the amine polymer is an aliphatic amine polymer.

32. The pharmaceutical composition of claim 25, wherein the amine polymer is colestipol.

33. The pharmaceutical composition of claim 31, wherein the amine polymer is an aliphatic amine polymer comprising one or more repeat units represented by a formula selected from the group consisting of: 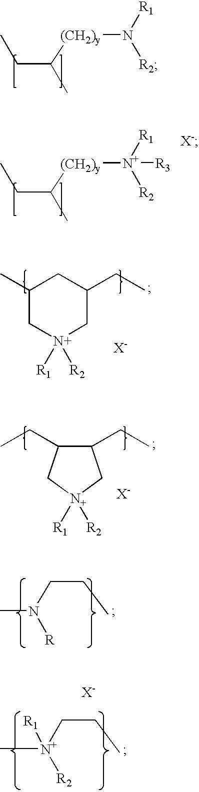
or a salt or a copolymer thereof, wherein:
y is zero or an integer from one to ten;
R, R₁, R₂ and R₃, independently, is H, a substituted or unsubstituted alkyl group or an aryl group; and
X⁻ is an exchangeable negatively charged counterion.

34. The pharmaceutical composition of claim 33, wherein the zinc salt is selected from the group consisting of zinc oxide, zinc acetate, zinc citrate, zinc carbonate and zinc tartrate.

35. The pharmaceutical composition of claim 34, wherein the zinc salt is zinc oxide.

36. The pharmaceutical composition of claim 34, wherein the aliphatic amine polymer is polyallylamine cross-linked by means of a multifunctional cross-linking agent.

37. The pharmaceutical composition of claim 36, wherein the polyallylamine is sevelamer.

38. (canceled)

39. A pharmaceutical composition, comprising:
a) a pharmaceutically acceptable carrier;
b) a pharmaceutically acceptable zinc salt; and
c) an agent selected from the group consisting of a pharmaceutically acceptable lanthanum salt, a pharmaceutically acceptable calcium salt, a pharmaceutically acceptable magnesium salt, a pharmaceutically acceptable iron salt, a phosphate transport inhibitor, an HMG-CoA reductase inhibitor and an alkaline phosphatase inhibitor.

40. (canceled)

41. The pharmaceutical composition of claim 39, wherein the pharmaceutically acceptable zinc salt is selected from the group consisting of zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine, zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate).

42. (canceled)

43. (canceled)

44. The pharmaceutical composition of claim 41, wherein the zinc salt is zinc oxide.

45. (canceled)

46. (canceled)
